(19) United States
(12) Patent Application Publication
(10) Pub. No.: US 2026/0273037 A1
(43) Pub. Date: Sep. 17, 2026

(54) ANTIBODIES

(71) Applicant: HYDROSENSE LTD, Linlithgow (GB)

(72) Inventors: Richard Campbell, Linlithgow (GB); Michael Connolly, Linlithgow (GB)

(73) Assignee: HYDROSENSE LTD, Linlithgow, GB (GB)

(21) Appl. No.: 19/166,966

(22) PCT Filed: Mar. 20, 2024
(86) PCT No.: PCT/EP2024/057502

§ 371 Date: Sep. 19, 2025

(30) Foreign Application Priority Data
Mar. 20, 2023 (GB) 2304025.6

(51) Int. Cl. A61K 39/02; A61P 37/04; C07K 16/126; G01N 33/543; G01N 33/569

(52) U.S. Cl. CPC A61K 39/0208; A61P 37/04; C07K 16/126; G01N 33/54388; G01N 33/56911; G01N 2333/195; G01N 2469/10

(57) ABSTRACT

The present invention is concerned with methods of producing antibodies specific against Legionella pneumophila SG (serogroups) 2-15, isolated and purified anti-Legionella pneumophila SG2-15 antibodies from same, and the antibodies for use in the detection of Legionella pneumophila (including in lateral flow and solid support tests). Also provided are the antibodies together with anti-Legionella pneumophila SG1 antibodies, the combined anti-SG1 and SG2-15 (i.e. SG1-15) antibodies for use in the detection of Legionella pneumophila, and methods of detection of Legionella pneumophila SG2-15 and SG1-15. Also provided are test kits for detecting the presence of Legionella pneumophila SG2-15 and Legionella pneumophila SG1-15.

### FIELD OF THE INVENTION

The present invention is concerned with methods of producing antibodies specific against Legionella pneumophila SG (serogroups) 2-15, isolated and purified anti-Legionella pneumophila SG2-15 antibodies from same, and the antibodies for use in the detection of Legionella pneumophila (including in lateral flow and solid support tests). Also provided are the antibodies together with anti-Legionella pneumophila SG1 antibodies, the combined anti-SG1 and SG2-15 (i.e. SG1-15) antibodies for use in the detection of Legionella pneumophila, and methods of detection of Legionella pneumophila SG2-15 and SG1-15. Also provided are test kits for detecting the presence of Legionella pneumophila SG2-15 and Legionella pneumophila SG1-15.

### BACKGROUND TO THE INVENTION

Legionella pneumophila is a species of Gram-negative bacteria that can cause a severe form of pneumonia called Legionnaires' disease. It is capable of colonising water systems such as domestic and commercial water systems, and presents a significant public health risk. The most common form of transmission of Legionella is inhalation of contaminated aerosols produced in conjunction with water sprays, jets or mists of contaminated water sources. This can include aerosols generated by showers, faucets, cooling towers, spas, and fountains.

The World Health Organisation (WHO) Legionellosis fact sheet (www.who.int/news-room/fact-sheets/detail/legionellosis) notes that of the reported cases, 75-80% are over 50 years of age, and 60-70% are male. It further notes that delay in diagnosis and administration of appropriate antibiotic treatment, increasing age and presence of co-existing diseases are predictors of death from Legionnaires' disease. The death rate may be as high as 40-80% in untreated immuno-suppressed patients and can be reduced to 5-30% through appropriate case management and depending on the severity of the clinical signs and symptoms. Overall, the death rate is usually within the range of 5-10%.

In Europe, the case fatality ratio is typically about 10% (European Centre for Disease Prevention and Control—ECDC—Surveillance Report “Legionnaire's disease in Europe 2013”—www.ecdc.europa.eu/sites/default/files/media/en/publications/Publications/legionnaires-disease-2015.pdf).

As noted in the WHO report, untreated Legionnaires' disease usually worsens during the first week. In common with other risk factors causing severe pneumonia, the most frequent complications of legionellosis are respiratory failure, shock and acute kidney and multi-organ failure. Therefore, rapid diagnosis and treatment of Legionnaires' disease (i.e. infection by Legionella pneumophila) can be extremely important.

There are currently 15 known serogroups of Legionella pneumophila, identified by differences in the composition of their cell wall antigens. Although serogroup 1 (SG1) is the most commonly isolated serogroup and is responsible for the majority of Legionnaire's disease cases, it is important to be able to test for the presence of the other serogroups (serogroups 2-15) (SG2-15).

As noted by the CDC (www.cdc.gov/Legionella/clinicians/diagnostic-testing.html), various categories of diagnostic test are available. Culture tests have a low sensitivity (20-80%) and high specificity (100%). However, their use requires the culturing of samples, which is slow and expensive. The Urinary Antigen Test (UAT) is rapid and has a relatively high sensitivity (70-100%) and specificity (95-100%). However, it can only detect serogroup 1 (SG1). PCR is highly sensitive and specific. However, it requires specialised lab equipment and PCR reagents (primers etc.), skilled staff, and is relatively expensive and slow (although not as slow as culture tests). DFA (Direct Fluorescence Antibody) stain has a relatively low sensitivity and a high specificity. Paired serology requires serum samples taken at multiple timepoints during infection (e.g. acute onset, 2 weeks after symptoms, and 3-6 weeks later).

Therefore, there is a strong need for simple, convenient, rapid test which can detect a wide range of Legionella pneumophila serogroups, in particular SG2-15. In order to achieve this, there is a need for suitable antibodies, and a suitable immunogen for the generation of such antibodies.

The present invention seeks to overcome at least one of the prior art disadvantages.

### SUMMARY OF THE INVENTION

According to a first aspect of the present invention, there is provided an immunogen for the production of anti-Legionella pneumophila serogroups 2-15 antibodies, comprising L. pneumophila serogroups 3, 4, 7, 8, 11, 13, and 15.

As detailed below, it has been found that using L. pneumophila serogroups (SG) 3, 4, 7, 8, 11, 13, and 15 to immunise animals results in a significant and unexpected improvement in production of antibodies specific against each of serogroups 2-15. This contrasts with other experiments performed (a) using fewer serogroups as immunogen, and (b) using more serogroups as immunogen.

Thus, the present invention provides an immunogen comprising L. pneumophila serogroups, the L. pneumophila serogroups consisting serogroups 3, 4, 7, 8, 11, 13, and 15. The immunogen can be for (i.e. suitable for) the production of antibodies specific against each of Legionella pneumophila serogroups 2-15.

Any reference herein to “anti-Legionella pneumophila serogroups 2-15 antibodies” is reference to antibodies specific against each of Legionella pneumophila serogroups 2-15. Similarly, any reference herein to “anti-Legionella pneumophila serogroup 1 antibody” is reference to antibody specific against Legionella pneumophila serogroup 1.

As per the experiments detailed below, the immunogen can additionally comprise a preservative. Examples of preservatives include bacteriostatic preservatives. Examples include sodium azide. Other examples are well known in the art and readily apparent to the person of ordinary skill.

Thus, in certain embodiments, the immunogen additionally comprises a preservative. In certain embodiments, the immunogen comprises a bacteriostatic preservative.

In certain embodiments, the immunogen additionally comprises a pharmaceutically acceptable carrier, diluent or excipient (Remington, The Science and Practice of Pharmacy, 2020, ISBN: 9780128200070).

Thus, the present invention can alternatively be viewed as providing (i.e. also provides) a pharmaceutical composition, the composition comprising an immunogen comprising L. pneumophila serogroups, the L. pneumophila serogroups consisting serogroups 3, 4, 7, 8, 11, 13, and 15. In certain embodiments, the pharmaceutical composition additionally comprises a pharmaceutically acceptable carrier, diluent or excipient. In certain embodiments the pharmaceutical composition additionally comprises a preservative. In certain embodiments, the pharmaceutical composition additionally comprises a bacteriostatic preservative.

The pharmaceutical composition can also be referred to as being a “pharmaceutical preparation”.

Unless the context dictates otherwise, reference herein to the immunogen is also reference to a pharmaceutical composition comprising the immunogen, i.e. aspects of the invention/features of the invention relating to the immunogen apply mutatis mutandis (i.e. are equally applicable) to the pharmaceutical composition.

As noted above, the majority of L. pneumophila infections are with serogroup 1, and that is what the standard rapid diagnostic assays are specific for. Diagnostic tests for other serogroups (i.e. one or more of serogroups 2-15) are typically more complex, costly (requiring complex reagents, expensive laboratory equipment, and energy usage in operating them), and time-consuming. The immunogens of the present invention and the antibodies which can be produced using them provide a significant improvement, including an environmental improvement insofar as energy, reagents, single-use plastics etc. from performing multiple tests for each of SG2-15 can be avoided with the provision of a single test for the presence of any of SG2-15.

In certain embodiments, the L. pneumophila serogroup 3 is serogroup 3 (Bloomington-2).

In certain embodiments, the L. pneumophila serogroup 4 is serogroup 4 (Los Angeles-1).

In certain embodiments, the L. pneumophila serogroup 7 is serogroup 7 (Chicago-8).

In certain embodiments, the L. pneumophila serogroup 8 is serogroup 8 (Concorde-3).

In certain embodiments, the serogroup 15 is serogroup 15 (Lansing-3).

In certain embodiments, the immunogen comprises L. pneumophila serogroups 3 (Bloomington-2), 4 (Los Angeles-1), 7 (Chicago-8), 8 (Concorde-3), 11, 13, and 15 (Lansing-3).

In certain embodiments, the L. pneumophila serogroup 3 (Bloomington-2) is the strain NCTC 11232. In certain embodiments, the L. pneumophila serogroup 4 (Los Angeles-1) is the strain NCTC 11233. In certain embodiments, the L. pneumophila serogroup 7 (Chicago-8) is the strain NCTC 11984. In certain embodiments, the L. pneumophila serogroup 8 (Concorde-3) is the strain NCTC 11985. In certain embodiments, the L. pneumophila serogroup 11 is the strain NCTC 12179. In certain embodiments, the L. pneumophila serogroup 13 is the strain NCTC 12181. In certain embodiments, the L. pneumophila serogroup 15 (Lansing-3) is the strain ATCC 35251.

In certain embodiments, the immunogen comprises L. pneumophila serogroups 3 (Bloomington-2 NCTC 11232), 4 (Los Angeles-1 NCTC 11233), 7 (Chicago-8 NCTC 11984), 8 (Concorde-3 NCTC 11985), 11 (NCTC 12179), 13 (NCTC 12181), and 15 (Lansing-3 ATCC 35251).

In certain embodiments, the L. pneumophila of the immunogen are dead, i.e. have been killed. Methods to kill the L. pneumophila forming the immunogen will be readily apparent to one of ordinary skill in the art. For example, they can be heat-killed, killed with a lysis buffer, or by sonication.

In certain embodiments, the immunogen of the present invention is for use in immunisation. In particular embodiments, the immunogen is for the production of anti-Legionella pneumophila serogroups 2-15 antibodies (i.e. antibodies specific against each of Legionella pneumophila serogroups 2-15) by immunisation.

In particular, immunisation can be of members of the Caprinae taxonomic group (sub-family) including sheep, domestic sheep (Ovis aries), and goats (Capra). In certain embodiments, immunisation is of sheep, more particularly domestic sheep. In certain embodiments, immunisation is of goats (Capra).

Also provided is a method of immunisation, comprising the step of administering the immunogen of the present invention to a subject. Subjects can include (but are not limited to) members of the Caprinae taxonomic group (sub-family) including sheep, domestic sheep (Ovis aries), and goats (Capra). In certain embodiments, immunisation is of sheep, more particularly domestic sheep. In certain embodiments, immunisation is of goats (Capra).

Also provided is a method of producing anti-Legionella pneumophila serogroups 2-15 antibodies (i.e. antibodies specific against each of Legionella pneumophila serogroups 2-15), the method comprising collecting antiserum from an animal which has been immunised with an immunogen according to the present invention.

The method can additionally comprise the step of isolating and/or purifying anti-Legionella pneumophila serogroups 2-15 antibodies (i.e. antibodies specific against each of Legionella pneumophila serogroups 2-15) from the antiserum. Suitable methods of isolation/purification include (but are not limited to) physicochemical fractionation (such as size exclusion chromatography, ammonium sulfate precipitation, ion exchange chromatography, immobilized metal chelate chromatography, and thiophilic adsorption), class-specific affinity purification (e.g. using Protein A, Protein G, or Protein L), and antigen-specific affinity purification (for example, using an affinity purification column with Legionella pneumophila lysate as the antigen) and other affinity chromatographic methods. In certain embodiments, the method comprises affinity purification using an affinity purification column with Legionella pneumophila lysate.

Also provided is anti-Legionella pneumophila serogroups 2-15 antibodies (i.e. antibodies specific against each of Legionella pneumophila serogroups 2-15) produced according to the above method or antigen binding fragments thereof.

In certain embodiments, the antibodies are isolated. In certain embodiments, the antibodies are purified. In certain embodiments, the antibodies are isolated and purified. Methods of isolation and purification of antibodies are well known in the art (e.g. Gottschalk U et al., Process Scale Purification of Antibodies, 2017, Wiley, ISBN: 9781119126911, doi: 10.1002/9781119126942).

Also provided is anti-Legionella pneumophila serogroups 2-15 antibodies or antigen binding fragments thereof, in combination with anti-Legionella pneumophila serogroup 1 antibody or an antigen binding fragment thereof. As detailed below, using the Legionella pneumophila serogroups (SG) 3, 4, 7, 8, 11, 13, and 15 is excellent for the production of anti-SG 2-15 antibodies. However, this does not result in the production of significant (or sufficient) serogroup 1 antibodies. Therefore, anti-Legionella pneumophila serogroups 1-15 antibodies or antigen binding fragments thereof can be produced by adding (i.e. combining with) a separate anti-Legionella pneumophila serogroup 1 antibody or an antigen binding fragment thereof.

Anti-Legionella pneumophila SG1 antibodies are well known in the art, e.g. Thermo Fisher Scientific catalog number: MA1-83288 (www.thermofisher.com), Abbexa Limited catalogue number: abx411454 (www.abbexa.com), QED Biopsciences Inc. catalog number: 15901 (www.qedbio.com).

Also provided is an immunoassay for detecting the presence of Legionella pneumophila, comprising antibodies or antigen binding fragments thereof according to the present invention (i.e. antibodies specific against each of Legionella pneumophila serogroups 2-15, or antigen binding fragments thereof). In certain embodiments, the immunoassay is for the detection of serogroups 2-15 only.

Also provided is an immunoassay for detecting the presence of Legionella pneumophila, comprising: (i) anti-Legionella pneumophila SG2-15 antibodies (i.e. antibodies specific against each of Legionella pneumophila serogroups 2-15) or antigen binding fragments thereof according to the present invention, and (ii) anti-Legionella pneumophila SG1 antibody (i.e. antibody specific against Legionella pneumophila serogroup 1) or an antigen binding fragment thereof.

In certain embodiments, the immunoassay is a lateral flow immunoassay, and comprises:
a first test line comprising antibody specific against Legionella pneumophila serogroup 1 or an antigen binding fragment thereof; and
a second test line comprising antibodies specific against each of Legionella pneumophila serogroups 2-15 or antigen binding fragments thereof, the antibodies produced according to the method of claim 12 or 13.

In certain embodiments, the immunoassay additionally comprises a control line.

Examples of immunoassays include diagnostic test kits.

A wide range of immunoassays are well known in the art, including competitive and non-competitive immunoassays, direct and indirect immunoassays, Western blots (immunoblotting), enzyme linked immunosorbent assay (ELISA) (including direct ELISA, indirect ELISA, sandwich ELISA and competitive ELISA), and bead-based immunoassays.

In certain embodiments, the immunoassay is a lateral flow immunoassay. In other embodiments, the immunoassay is an ELISA.

In certain embodiments, the anti-Legionella pneumophila antibodies are immobilised on a surface. In certain embodiments, they are covalently bound to the surface. In certain embodiments, they are directly covalently bound to the surface. In other embodiments, they are indirectly covalently bound to the surface.

In the case of a lateral flow immunoassay, it can be provided with at least two test lines-one test line for L. pneumophila serogroup 1, and one test line for L. pneumophila serogroups 2-15. The anti-Legionella pneumophila serogroups 2-15 antibodies or antigen binding fragments thereof can be provided as the “capture antibody” for the L. pneumophila serogroups 2-15 test line. The anti-Legionella pneumophila serogroup 1 antibody or antigen binding fragment thereof can be provided as the “capture antibody” for the L. pneumophila serogroups 1 test line. Such lateral flow immunoassays can additionally comprise a control line, the control in-use acting to confirm that the immunoassay has run properly (that reagent has migrated/passed from a sample inlet across the lateral flow substrate and the first and second test lines to the control line).

An exemplary lateral flow immunoassay for L. pneumophila SG1 and SG2-15 is illustrated in FIGS. 12A-12D. An exemplary lateral flow immunoassay for L. pneumophila SG1-15 is illustrated in FIGS. 13A-C.

Also provided according to the present invention is a method of detecting the presence of Legionella pneumophila in a sample, the method comprising the steps of:
(i) contacting the sample with anti-Legionella pneumophila antibodies (i.e. antibodies specific against each of Legionella pneumophila serogroups 2-15) or antigen binding fragments thereof according to the present invention;
(ii) detecting any antibody-antigen binding reaction; and
(iii) correlating the results of detection step (ii) with the presence of Legionella pneumophila.

Thus, a negative result (i.e. no detected antibody-antigen binding reaction) can indicate that (is indicative that) Legionella pneumophila is not present in the sample, i.e. indicates (is indicative of) an absence of Legionella pneumophila in the sample.

In certain embodiments, it is a method for detecting the presence of Legionella pneumophila serogroups 2-15, and the antibodies or antigen binding fragments thereof are specific against Legionella pneumophila serogroups 2-15, i.e. are the anti-Legionella pneumophila serogroups 2-15 antibodies or antigen binding fragments of the present invention.

In other embodiments, it is a method for detecting the presence of Legionella pneumophila serogroups 1-15, and the antibodies or antigen binding fragments thereof are specific against Legionella pneumophila serogroups 1-15, i.e. are the anti-Legionella pneumophila serogroups 2-15 antibodies or antigen binding fragments of the present invention, together with an anti-Legionella pneumophila serogroup 1 antibody or an antigen binding fragment thereof.

The term “comprising” as used herein to specify the inclusion of components also includes embodiments in which no further components are present.

Particular and preferred aspects of the invention are set out in the accompanying independent claims. Combinations of features from the dependent claims may be combined with features of the independent claims as desired and appropriate and not merely as explicitly set out in the claims.

### DRAWINGS

FIG. 1 shows results of titration of naïve sheep serum against antigen (QC Sample C (mixture of heat-killed potentially cross-reacting bacteria);

FIG. 2 shows results of titration of naïve sheep serum against antigen (QC Sample E (mixture of heat-killed non-L. pneumophila SG1);

FIG. 3 shows results of titration of naïve sheep serum against antigen (QC Sample N (heat killed L. pneumophila SG1);

FIG. 4 shows titres from antiserum assessment 1 (antigen: Immunogen 1.1);

FIG. 5 shows titres from antigen assessment 2 (antigen: Immunogen 1.1)

FIG. 6 shows Test Bleed 1 reactivity to SG1-15 serogroups, non-pneumophila and non-Legionella spp mix;

FIG. 7 shows Test Bleed 3 reactivity to SG1-15 serogroups, non-pneumophila and non-Legionella spp mix;

FIG. 8 shows Test Bleed 5 reactivity to SG1-15 serogroups, non-pneumophila and non-Legionella spp mix;

FIG. 9 shows reaction profile of Program 1 Sheep 1 & 2 (final bleed) compared to Program 2 Sheep 3 (initial bleed) by direct ELISA;

FIG. 10 shows the reactivity profile for the SG2-15 (Immunogen 2.1) immunised sheep from Program 2;

FIG. 11 shows that reactivity profile for the SG2-15 (Immunogen 2.1) immunised sheep (Sheep 3) from Program 2 compared to SG2-15 (Immunogen 2.1) immunised sheep (Sheep 4) from Program 3;

FIG. 12A-D show lateral flow immunoassay test devices for L. pneumophila SG1-15. FIG. 12A—unused test device. FIG. 12B—positive test for SG1. FIG. 12C—positive test for SG2-15. FIG. 12D—negative test for SG1 and SG2-15 (i.e. negative test for SG1-15); and

FIG. 13A-C show lateral flow immunoassay test devices for L. pneumophila SG1-15. FIG. 12A—unused test device. FIG. 12B—positive test for SG1-15. FIG. 12C—negative test for SG1-15.

### SPECIFIC EMBODIMENTS

A fully and enabling disclosure of the present invention, including the best mode thereof, to one of ordinary skill in the art, is set forth more particularly in the remainder of the specification. Reference now will be made in detail to the embodiments of the invention, one or more examples of which are set forth below. Each example is provided by way of explanation of the invention, not limitation of the invention.

It will be apparent to those of ordinary skill in the art that various modifications and variations can be made in the present invention without departing from the scope of the invention. For instance, features described as part of one embodiment can be used on another embodiment to yield a still further embodiment. Thus, it is intended that the present invention cover such modifications and variations as come within the scope of the appended claims and their equivalents.

Other objects, features, and aspects of the present invention are disclosed in the remainder of the specification. It is to be understood by one of ordinary skill in the art that the present discussion is a description of exemplary embodiments only and is not intended as limiting the broader aspects of the present invention, which broader aspects are embodied in the exemplary constructions.

### Experiments

### SUMMARY

### Initial Serum Screening

Prior to any immunisation being undertaken, serum from sheep was screened (“Serum screening” section, below) for indigenous levels of antibody against three antigens, including L. pneumophila. None of the tested sheep showed native immunity to the antigens.

### Program 1

Aim: To raise antibodies against SG2-15.

In a first experiment (Program 1), a first sheep (Sheep 1) was immunised with a first immunogen (Immunogen 1.1) comprising heat-killed L. pneumophila SG 3 (Bloomington-2 NCTC 11232), 5 (Dallas-1E NCTC 11405), and 6 (Oxford NCTC 11287). Results showed that a desired level of reactivity was not achieved across each of serogroup 2-15.

In a second experiment (Program 1), a second sheep (Sheep 2) was immunised using a second immunogen (Immunogen 1.2) comprising heat-killed L. pneumophila SG 3 (Bloomington-2 NCTC 11232), 5 (Dallas-1E NCTC 11405), and 6 (Oxford NCTC 11287, together with L. pneumophila SG2 (Togus-1 NCTC 11230), 4 (Los Angeles-1 NCTC 11233), 7 (Chicago-8 NCTC 11984), 8 (Concorde-3 NCTC 11985), 10 (LEIDEN-1 NCTC 12000), 11 (NCTC 12179), 13 (NCTC 12181), and 15 (Lansing-3 ATCC 35251), i.e. targeting the serogroups for which sufficient reactivity was not observed in the first experiment. Results showed that the reactivity profile achieved across all serogroup did not change significantly.

### Program 2

In a third experiment (Program 2), a sheep (Sheep 3) was immunised using an immunogen (Immunogen 2.1) comprising heat-killed L. pneumophila SG 3 (Bloomington-2 NCTC 11232), 4 (Los Angeles-1 NCTC 11233), 7 (Chicago-8 NCTC 11984), 8 (Concorde-3 NCTC 11985), 11 (NCTC 12179), 13 (NCTC 12181), and 15 (Lansing-3 ATCC 35251). Screening of serum obtained from this sheep showed that reactivity to each of serogroups 2-15 was improved (not just the serogroups contained in the second immunogen mix) compared to results obtained with the Program 1 experiments.

### Program 3

In a fourth experiment (Program 3), another sheep (Sheep 4) was immunised with Immunogen 2.1. Serum obtained from the host was screened. Results showed that the reactivity to SG2-15 was the same as that for the serum obtained from the Program 2 hosts. Thus, the immunogenicity of Immunogen 2.1 is not dependent upon the host animal (i.e. the animal that is immunised with the immunogen).

### Methods

### Microorganism Depositary Institutions:

ATCC-American Type Culture Collection, 10801 University Boulevard, Manassas, Virginia 20110-2209, USA; www.atcc.org
NCIMB-NCIMB Ltd, Wellheads Place, Dyce, Aberdeen, AB21 7 GB, UK; www.ncimb.com
NCTC-National Collection of Type Cultures, UK Health Security Agency, Porton Down, Salisbury, SP4 0JG, UK; www.culturecollections.org.uk

### Program 1

### Immunogen 1.1

For a first attempt to raise antibodies against SG2-15, a first immunogen (Immunogen 1.1) was prepared as follows:
L. pneumophila serogroups 3 (Bloomington-2 NCTC 11232), 5 (Dallas-1E NCTC 11405), and 6 (Oxford NCTC 11287) were cultured and heat-killed by Express Micro (Express Micro Science (Express Microbiology Ltd.), Unit 22/4, Mill Road Industrial Estate, Linlithgow, West Lothian, Scotland, UK EH49 7SF; www.expressmicroscience.co.uk) as follows:
   i. From frozen stocks of each of the L. pneumophila serogroups (above), bacteria were cultured onto BCYE (buffered charcoal yeast extract) agar for 5 days. A further subculture was performed onto fresh BCYE agar (×10 plates). This subculture was grown up for 5 days also.
   ii. A heavy solution of the bacteria was made up by emulsifying cultures into 10 ml of autoclaved Ringers solution in a heat proof bottle and mixed well.
   iii. A small aliquot (~1 ml) of live solution was removed, labelled and set aside.
   iv. The remaining solution was heat treated in a water bath at 90° C. for 30 minutes with regular agitation. The solution was then removed.
   v. Once the solution was cool, step iv was repeated.
   vi. To verify all bacteria had been killed, 0.5 mL was plated out in triplicate onto BCYE agar. Plates were incubated for 10 days to confirm no growth and therefore samples were verified as dead.
95 μL of 10% sodium azide solution was added to each 10 mL heat-killed solution, giving a final sodium azide concentration of 0.095%.

A 4 ml sample from each heat-killed solution (i.e. of each of L. pneumophila SG 3 (Bloomington-2 NCTC 11232), 5 (Dallas-1E NCTC 11405), and 6 (Oxford NCTC 11287)) was added to a fresh 15 mL Falcon® tube and vortexed to mix.

The tube was labelled and stored at 2-8 Deg C.

### Immunogen 1.2

As detailed below, results obtained when immunising sheep with Immunogen 1.1 showed inadequate immunogenicity across serogroups 2-15. Therefore, part-way through the vaccination schedule (see the “Immunisation” section below), the immunogen for both sheep was switched across to Immunogen 1.2.

To prepare the immunogen to raise antibodies against missing SG2-15 (see results obtained using Immunogen 1.1), Immunogen 1.2 was prepared as follows:

Heat-killed solutions of each of L. pneumophila SG 3 (Bloomington-2 NCTC 11232), 5 (Dallas-1E NCTC 11405), and 6 (Oxford NCTC 11287) were prepared as above. A 1 in 10 dilution of each SG solution was performed in dH₂O, and 150 μL of each dilution was added to a 30 mL plastic universal container.

Heat-killed solutions of each of L. pneumophila SG2 (Togus-1 NCTC 11230), 4 (Los Angeles-1 NCTC 11233), 7 (Chicago-8 NCTC 11984), 8 (Concorde-3 NCTC 11985), 10 (LEIDEN-1 NCTC 12000), 11 (NCTC 12179), 13 (NCTC 12181), and 15 (Lansing-3 ATCC 35251) were prepared by Express Micro using the same protocol as for the preparation of the heat-killed solutions of SG 3, SG 5 and SG 6 (above).

The following volume of each sample was added to the 30 mL universal container (already containing the SG3, 5 and 6 dilutions).

TABLE 1.1

| L. pneumophila serogroup | Volume added (mL) |
|---|---|
| 2 | 2.25 |
| 4 | 2.25 |
| 7 | 1.50 |
| 8 | 1.50 |
| 10 | 2.55 |
| 11 | 1.50 |
| 13 | 1.50 |
| 15 | 1.50 |

142.5 μL of 10% sodium azide solution was added to the 30 ml universal container, giving a final sodium azide concentration of 0.095%.

The universal container was then vortexed and a 10 ml sample removed to a fresh 15 mL Falcon tube. The remaining volume was placed in a 15 mL Falcon tube.

Each tube was labelled and stored at 2-8 Deg C.

### Immunisation

Two sheep (animals: CF2244 and CF2245) were immunised as shown in Table 1.2 (both were immunised with the same immunogens at the same time points). Immunogen 1.1 was administered (as shown in Table 1.2) to both animals during the period from weeks 1-19. After week 19, Immunogen 1.2 was administered (as shown in Table 1.2) to both animals.

TABLE 1.2

| Week Number | 1 | 2 | 3 | 4 | 5 | 6 | 7 | 8 | 9 | 10 |
|---|---|---|---|---|---|---|---|---|---|---|
| Sheep 1 (CF2244) | P/S | | | | R | | S | | R | |
| Sheep 2 (CF2245) | P/S | | | | R | | S | | R | |
| Week Number | 11 | 12 | 13 | 14 | 15 | 16 | 17 | 18 | 19 | 20 |
| Sheep 1 (CF2244) | S | | R | | B | | R | | B | |
| Sheep 2 (CF2245) | S | | R | | B | | R | | B | |
| Week Number | 21 | 22 | 23 | 24 | 25 | 26 | 27 | 28 | 29 | 30 |
| Sheep 1 (CF2244) | | | R | | S | | R | | S | |
| Sheep 2 (CF2245) | | | R | | S | | R | | S | |
| Week Number | 31 | 32 | 33 | 34 | 35 | 36 | 37 | 38 | 39 | 40 |
| Sheep 1 (CF2244) | R | | S | | R | | S | | R | |
| Sheep 2 (CF2245) | R | | S | | R | | S | | R | |
| Week Number | 41 | 42 | 43 | 44 | 45 | 46 | 47 | 48 | 49 | 50 |
| Sheep 1 (CF2244) | S | | R | | B | | R | | B | |
| Sheep 2 (CF2245) | S | | R | | B | | R | | B | |
| Week Number | 51 | 52 | 53 | 54 | 55 | 56 | 57 | 58 | 59 | 60 |
| Sheep 1 (CF2244) | R | | B | | R | | B | | | |
| Sheep 2 (CF2245) | R | | B | | R | | B | | | |

Key:
P—Primary immunisation
R—Re-immunisation
S—Sample
B—Bleed

### Serum Screening

Serum from six sheep (Samples 1-6) was screened for indigenous levels of antibody against three antigens to select the most suitable sheep for use in production of Legionella antisera.

### Method

1. Plates were coated as per Table 1.3:

TABLE 1.3

| Plate No. | Antigen (Ag) | Dilution | Volume/well | Incubation |
|---|---|---|---|---|
| 1-2 | QC Sample C | 1/1000 | 100 μL | 4 DegC. overnight |
| 3-4 | QC Sample E | 1/1000 | 100 μL | 4 DegC. overnight |
| 5-6 | QC Sample N | 1/1000 | 100 μL | 4 DegC. overnight |

QC Sample C: Mixture of heat killed potentially cross-reacting bacteria, as detailed below:

| Bacterium | Strain | Isolate number |
|---|---|---|
| Pseudomonas fluorescens | Migula 1895 (AL) | NCTC 10038 |
| Burkholderia cepacian | Sneath D338 | NCIMB 8507 |
| Klebsiella oxytoca | 641 | NCIMB 2121 |
| Ralstonia pickettii | PockettK-288 | NCIMB 13142 |
| | Kosako85011 | |
| Raoultella terrigena | — | NCIMB 8135 |
| Escherichia coli | — | ATCC 8739 |
| Citrobacter freundii | — | ATCC 8090 |
| Yersinia ruckeri | RM3 | NCTC 12986 |
| Pseudomonas aeruginosa | Schroeter 1872 | NCTC 1032 |
| | Migula 1900 | |
| Enterobacter cloacea | | ATCC 3530 |
| Acinetobacter calcoaceticus | Bouvet & Girmont | NCTC 12983 |
| | doudoro ff46 | |
| Pseudomonas putida | B-12 | NCIMB 18 |
| Aeromonas hydrophila | BPE 143 | NCTC 8049 |
| | | (ATCC 7966) |
| Pseudomonas stutzeri | H. Lautrop | NCTC 12262 |
| | AB 201 | (ATCC 17588) |
| Bacillus subtilis | — | ATCC 6633 |
| Streptococcus pyogenes | GP A | NCTC 12696 |
| Citrobacter koseri | 14804 | NCTC 10786 |
| | | (ATCC 27028) |
| Serratia marcessens | — | NCIMB 10211 |
| Proteus mirablis | — | NCIMB 11938 |
| Staphylococcus epidermidis | — | NCIMB 11047 |

QC Sample E: Mixture of heat-killed L. pneumophila SG2-15.
QC Sample N: Heat-killed L. pneumophila SG1
Antigen was diluted in PBS (phosphate buffered saline) prior to coating plates.
2. Plates washed 3 times with PBS +0.05% Tween 20.
3. Plates blocked with 300 μL/well PBS +0.05% Tween 20+2% BSA (bovine serum albumin) for 1 hour at 37 Deg C.
4. Plates washed 3 times with Tris +0.05% Tween 20.
5. Serum samples from six sheep diluted 1/500 in PBS +0.05% Tween 20 then ½ down the plate (100 μL sample+100 μL PBS +0.05% Tween 20).
   Control antibodies diluted 1/500 in PBS +0.05% Tween 20 and diluted ½ down plate.
   Plates incubated for 1 hour at 37 Deg C.
6. Plates washed 3 times in Tris +0.05% Tween 20.
7. Secondary Ab diluted 1/30000 in PBS +0.05% Tween 20 and 100 μL added per well.
   Plates incubated for 1 hour at 37 Deg C.
8. Plates washed 3 times in PBS +0.05% Tween 20.
9. 100 μL TMB (3,3′,5,5′-Tetramethylbenzidine) added per well and incubated at 37 Deg C. for 15 minutes.
10. Reaction stopped with 100 μL H₂SO₄. (1.0M).
11. Plates read at 450 nm.
“Sero 1” (see Figs) is 100 mM Phosphate Buffered Saline pH 7.4, Sigma P4417.

### Results:

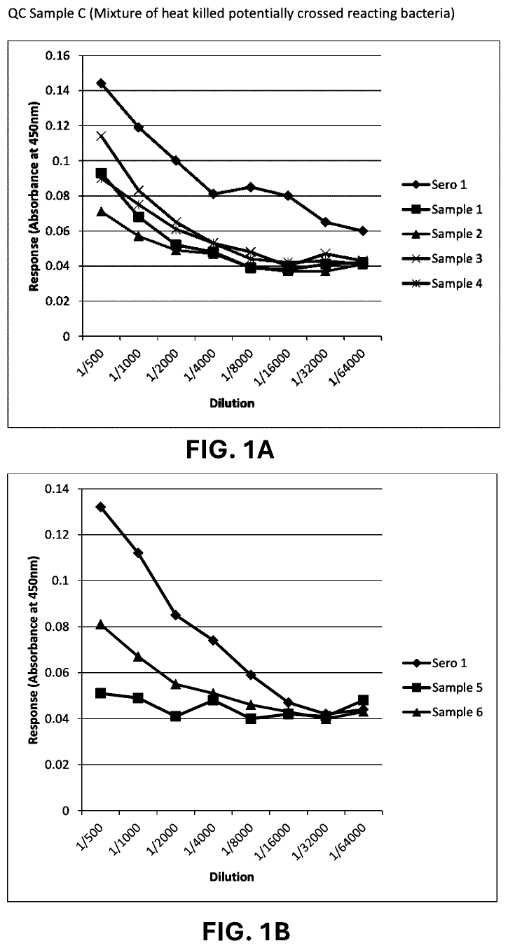

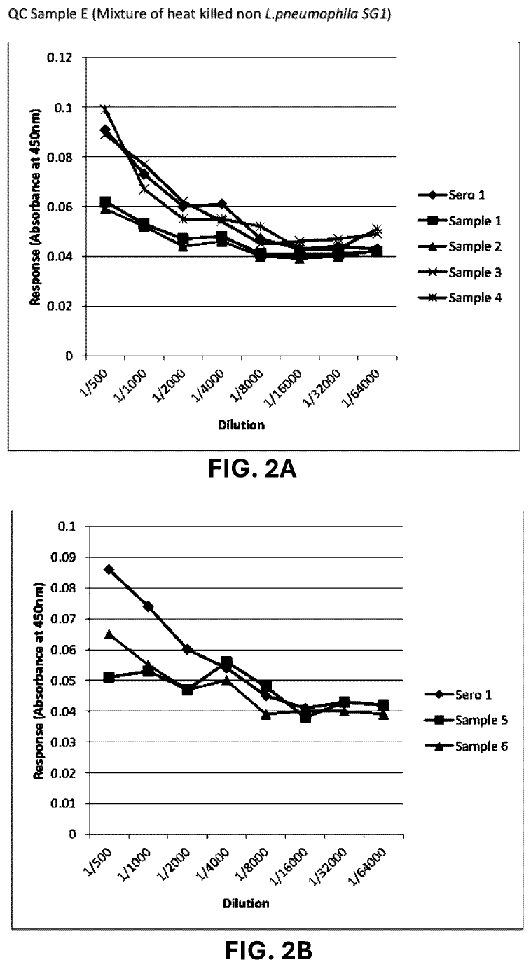

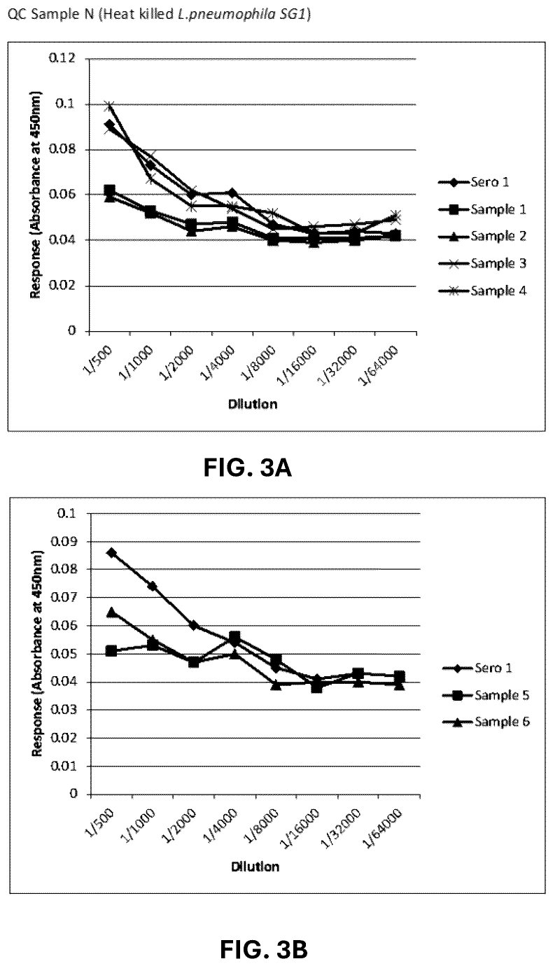

Results are shown in FIGS. 1, 2 and 3.

### Conclusions

All the sheep screened showed little activity to each antigen tested, and results were comparable to the background signal.

### Antiserum Assessment 1

Antigen: L. pneumophila SG 3, 5, 6 (Immunogen 1.1)

ELISA format: Indirect ELISA format with end point titre determined as the dilution at which the positive response is equivalent to the negative response. 50% titre is determined as the dilution at which the response is reduced by half.

### Calculation of Titre:

TABLE 1.4

| | Weeks post primary | | |
|---|---|---|---|
| Sheep number | immunisation | 50% Titre | End Point Titre |
| CF2244 | 6 | 19958 | >1/64000 |
| CF2245 | 6 | 36588 | >1/64000 |

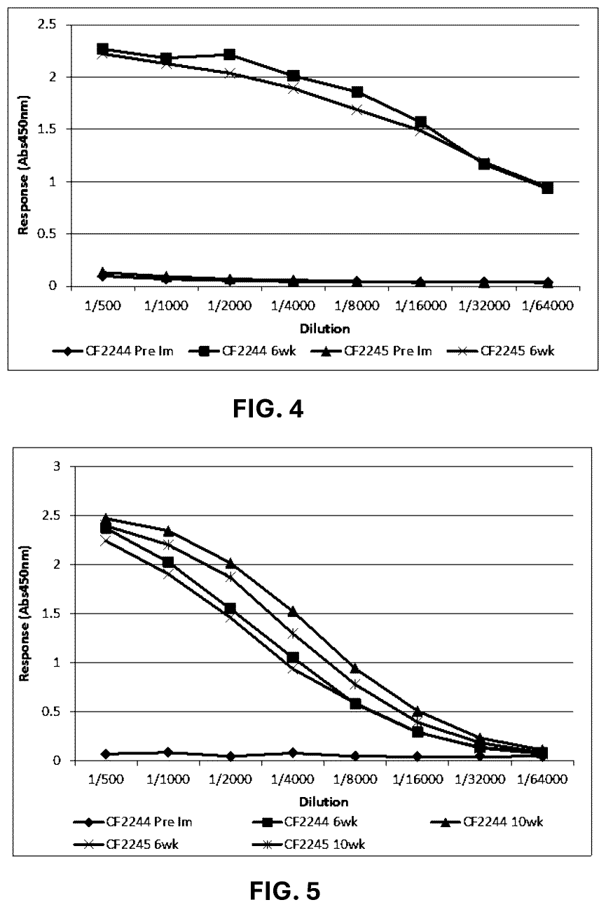

Antibody Titration Curves: Shown in FIG. 4. “Pre Im” curves are pre-immunisation. “6 wk” curves are for 6 weeks post primary immunisation.

Results: The results indicate that both sheep gave good response against L. pneumophila SG 3, 5 and 6 after 6 weeks.

### Antiserum Assessment 2

Antigen: L. pneumophila SG 3, 5, 6 (Immunogen 1.1)

ELISA format: Indirect ELISA format with end point titre determined as the dilution at which the positive response is equivalent to the negative response. 50% titre is determined as the dilution at which the response is reduced by half.

### Calculation of Titre:

TABLE 1.5

| | Weeks post primary | | |
|---|---|---|---|
| Sheep number | immunisation | 50% Titre | End Point Titre |
| CF2244 | 10 | 81047 | >1/4374000 |
| CF2245 | 10 | 57667 | >1/4374000 |

Antibody Titration Curves: Shown in FIG. 5. “Pre Im” curves are pre-immunisation. “10 wk” curves are for 10 weeks post primary immunisation.

Results: The results indicate that both sheep gave good response against L. pneumophila SG 3, 5 and 6 after 10 weeks.

Following the 10-week titre check, further ELISAs were performed using the same method as described above with the exception that wells were coated with individual heat killed serogroups 1-15 so as to observe reactivity for each. The heat killed serogroup suspensions were normalised by measuring the absorbance of the solution at 280 nm and diluting/concentrating the suspension until OD=1 at 280 nm. This was then diluted in PBS (phosphate buffered saline) prior to coating the plate.

### Results:

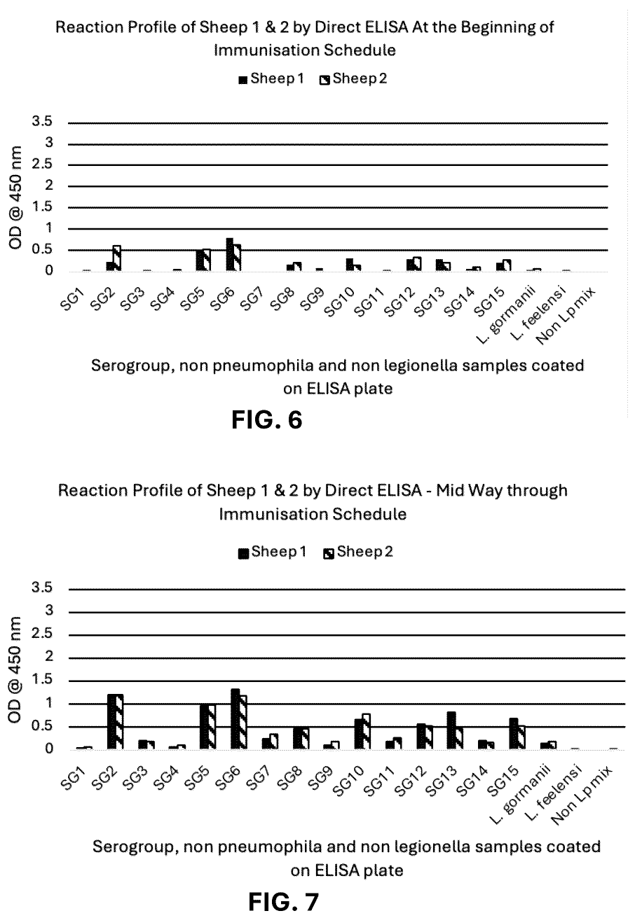

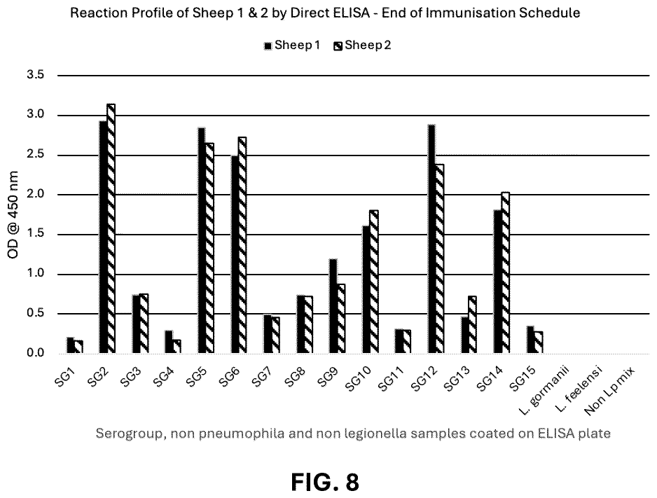

FIG. 6 shows Test Bleed 1 reactivity to SG1-15 serogroups, non-pneumophila and non-Legionella spp mix. FIG. 7 shows Test Bleed 3 reactivity to SG1-15 serogroups, non-pneumophila and non-Legionella spp mix. FIG. 8 shows Test Bleed 5 reactivity to SG1-15 serogroups, non-pneumophila and non-Legionella spp mix.

(In the Figs.: L. gomanii is Legionella gomanii; L. feelensi is Legionella feelensi, i.e. non-pneumophila Legionella species)

### Conclusions

FIGS. 6-8 show that there was a gradual increase in reactivity across serogroups 1-15 over the immunisation schedule but several serogroups (SG3, 4, 7, 8, 9, 11, 13 and 15) had reduced reactivity as measured by ELISA. The immunogen was switched from Immunogen 1.1 to Immunogen 1.2 during the schedule, but the reactivity profile didn't change i.e., the lower reactive serogroups did not increase in reactivity.

### Program 2

### Immunogen 2.1—SG2-15 Polyclonal Antibody Generation

In a first attempt to raise antibodies against missing SG2-15 in a new host, it was decided to: (i) replace SG2 with SG3, and (ii) not use SG10. An immunogen (Immunogen 2.1) was therefore prepared as follows:

L. pneumophila SG 3 (Bloomington-2 NCTC 11232), 4 (Los Angeles-1 NCTC 11233), 7 (Chicago-8 NCTC 11984), 8 (Concorde-3 NCTC 11985), 11 (NCTC 12179), 13 (NCTC 12181), and 15 (Lansing-3 ATCC 35251) were prepared by Express Micro using the same protocol as for the preparation of the heat-killed solutions of SG 3, SG 5 and SG 6 (above).

The following volume of each sample was added to a 30 mL universal container.

TABLE 2.1

| L. pneumophila serogroup | Volume added (mL) |
|---|---|
| 3 | 2.5 |
| 4 | 2.5 |
| 7 | 2.5 |
| 8 | 2.5 |
| 11 | 2.5 |
| 13 | 2.5 |
| 15 | 2.5 |

166.7 μL of 10% sodium azide solution was added to the 30 mL universal container to give a final sodium azide concentration of 0.095%.

The universal container was vortexed and a 10 ml sample removed to a fresh 15 mL Falcon tube. The remaining volume was placed in a 15 mL Falcon tube.

Each tube was labelled and stored at 2-8 Deg C.

Sheep 3 was immunised with Immunogen 2.1 (SG2-15).

Immunisation schedule is as shown in Table 2.2:

TABLE 2.2

| Week Number | 1 | 2 | 3 | 4 | 5 | 6 | 7 | 8 | 9 | 10 |
|---|---|---|---|---|---|---|---|---|---|---|
| Sheep 3 (CF2281) | S/P | | | | R | | S | | R | |
| Week Number | 11 | 12 | 13 | 14 | 15 | 16 | 17 | 18 | 19 | 20 |
| Sheep 3 (CF2281) | B | | R | | B | | R | | B | |
| Week Number | 21 | 22 | 23 | 24 | 25 | 26 | 27 | 28 | 29 | 30 |
| Sheep 3 (CF2281) | R | | B | | R | | B | | R | |
| Week Number | 31 | 32 | 33 | 34 | 35 | 36 | 37 | 38 | 39 | 40 |
| Sheep 3 (CF2281) | B | | R | | B | | R | | B | |
| Week Number | 41 | 42 | 43 | 44 | 45 | 46 | 47 | 48 | 49 | 50 |
| Sheep 3 (CF2281) | R | | B | | R | | B | | R | |
| Week Number | 51 | 52 | 53 | 54 | 55 | 56 | 57 | 58 | 59 | 60 |
| Sheep 1 (CF2244) | B | | R | | B | | R | | B | |
| Week Number | 61 | 62 | 63 | 64 | 65 | 66 | 67 | 68 | 69 | 70 |
| Sheep 3 (CF2281) | R | | B | | R | | B | | R | |
| Week Number | 71 | 72 | 73 | 74 | 75 | 76 | 77 | 78 | 79 | 80 |
| Sheep 3 (CF2281) | B | | R | | B | | R | | B | |
| Week Number | 81 | 82 | 83 | 84 | 85 | 86 | 87 | 88 | 89 | 90 |
| Sheep 3 (CF2281) | R | | B | | R | | B | | R | |

| Week Number | 91 | 92 | 93 | 94 | 95 |
|---|---|---|---|---|---|
| Sheep 3 (CF2281) | B | | R | | B |

Key:
P—Primary immunisation
R—Re-immunisation
S—Sample
B—Bleed

Serum was screened using ELISAs performed using the same method as described above with wells being coated with individual heat killed serogroups 1-15 so as to observe reactivity for each. The heat killed serogroup suspensions were normalised by measuring the absorbance of the solution at 280 nm and diluting/concentrating the suspension until OD=1 at 280 nm. This was then diluted in PBS (phosphate buffered saline) prior to coating the plate.

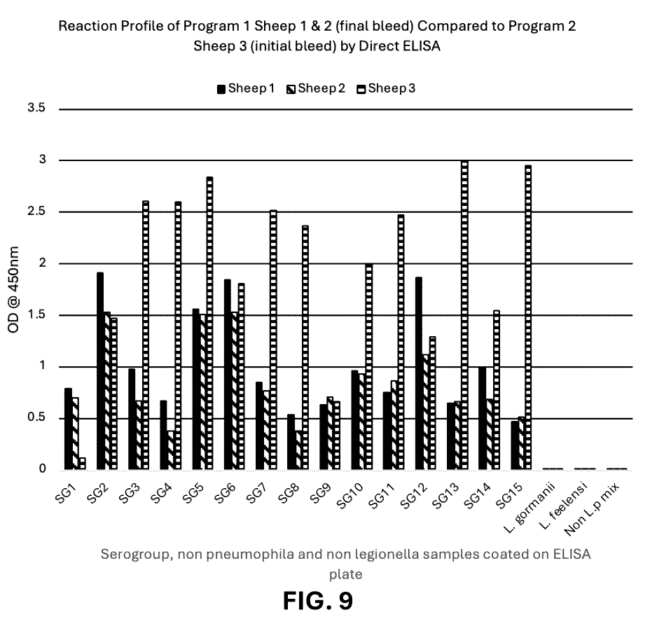

FIG. 9 shows that the new immunogen (Immunogen 2.1) generally gave much better reactivity across the serogroups than Immunogens 1.1 and 1.2.

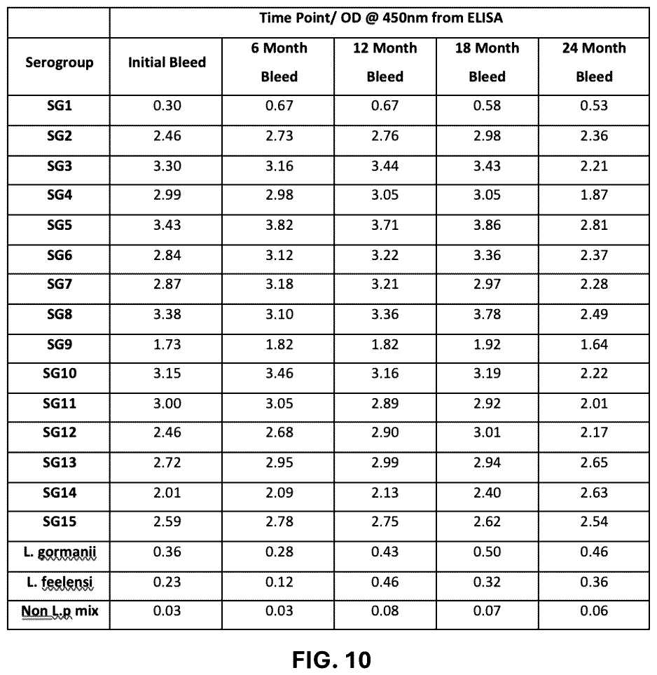

FIG. 10 shows the reactivity profile for the SG2-15 (Immunogen 2.1) immunised sheep from Program 2 across the schedule. Low reactivity to SG1, non-pneumophila and non-Legionella mix compared to reactivity to L. pneumophila SG2-15.

### Program 3

Due to sheep becoming ill during continued Program 2 immunisations, a third program was started to replace the SG2-15 sheep (Sheep 3).

The immunogen from Program 2 were used in Program 3, i.e. Immunogen 2.1 (SG2-15) was used to immunise one sheep (Sheep 4).

Immunisation schedule is as per Table 3.1.

TABLE 3.1

| Week Number | 1 | 2 | 3 | 4 | 5 | 6 | 7 | 8 | 9 | 10 |
|---|---|---|---|---|---|---|---|---|---|---|
| Sheep 4 (CF2352) | S/P | | | | R | | S | | R | |
| Week Number | 11 | 12 | 13 | 14 | 15 | 16 | 17 | 18 | 19 | 20 |
| Sheep 4 (CF2352) | B | | R | | B | | R | | B | |
| Week Number | 21 | 22 | 23 | 24 | 25 | 26 | 27 | 28 | 29 | 30 |
| Sheep 4 (CF2352) | R | | B | | R | | B | | R | |
| Week Number | 31 | 32 | 33 | 34 | 35 | 36 | 37 | 38 | 39 | 40 |
| Sheep 4 (CF2352) | B | | R | | B | | R | | B | |
| Week Number | 41 | 42 | 43 | 44 | 45 | 46 | 47 | 48 | 49 | 50 |
| Sheep 4 (CF2352) | R | | B | | R | | B | | R | |
| Week Number | 51 | 52 | 53 | 54 | 55 | 56 | 57 | 58 | 59 | 60 |
| Sheep 4 (CF2352) | B | | R | | B | | R | | B | |

| Week Number | 61 | 62 | 63 | 64 | 65 | 66 | 67 |
|---|---|---|---|---|---|---|---|
| Sheep 4 (CF2352) | R | | B | | R | | B |

Key:
P—Primary immunisation
R—Re-immunisation
S—Sample
B—Bleed

### Serum Screening is as for Program 2.

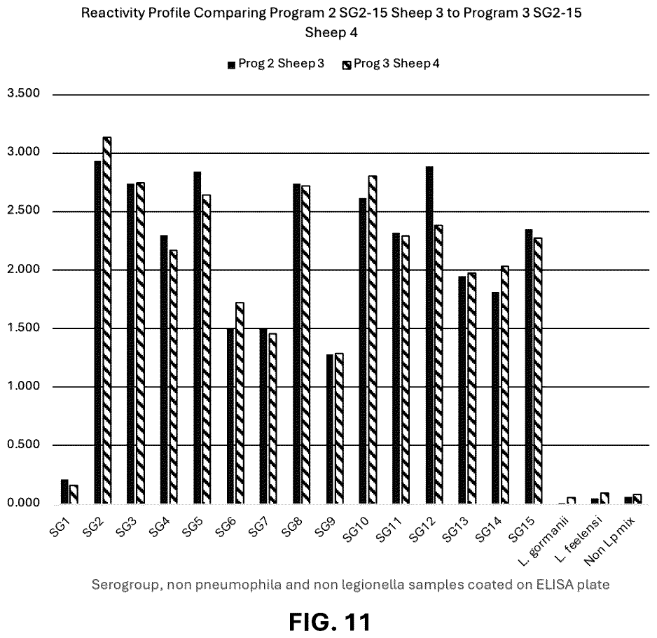

FIG. 11 shows that reactivity profile for the SG2-15 (Immunogen 2.1) immunised sheep (Sheep 3) from Program 2 compared to SG2-15 (Immunogen 2.1) immunised sheep (Sheep 4) from Program 3. It shows that having different hosts does not significantly impact the reactivity profile when testing by ELISA, i.e. Immunogen 2.1 gives generally/acceptably consistent (repeatable) results when used to immunise different animals.

### Conclusions

The use of Immunogen 2.1 has allowed for the generation of polyclonal antibodies specific against Legionella pneumophila serogroups 2-15 (SG2-15). This contrasts with the results obtained with Immunogen 1.1 (L. pneumophila SG 3 (Bloomington-2), 5 (Dallas-1E) and 6 (Oxford)) and Immunogen 1.2 (L. pneumophila SG 3 (Bloomington-2), 5 (Dallas-1E) and 6 (Oxford), together with L. pneumophila SG2 (Togus-1), 4 (Los Angeles-1), 7 (Chicago-8), 8 (Concorde-3), 10, 11, 13, and 15 (Lansing-3)). The efficacy of Immunogen 2.1 is unexpected, given that the only L. pneumophila serogroups it includes are L. pneumophila SG 3 (Bloomington-2), 4 (Los Angeles-1), 7 (Chicago-8), 8 (Concorde-3), 11, 13, and 15 (Lansing-3).

The resulting polyclonal anti-SG2-15 antibodies (e.g. in the form of an isolated and purified anti-SG2-15 antiserum) can be used in antibody-based tests, i.e. immunoassays.

Suitable immunoassays include (but are not limited to) lateral flow tests (LFTs) (lateral flow immunoassays), enzyme-linked immunosorbent assays, and solid support-based immunoassays.

### Isolation and Purification of Antibodies

Antibodies are isolated and purified from animals immunised with Immunogen 2.1 using an using an affinity purification column with Legionella pneumophila SG2-15 lysate as the antigen.

Suitable affinity purification protocols are well known to the person of ordinary skill in the art.

### Immunoassays

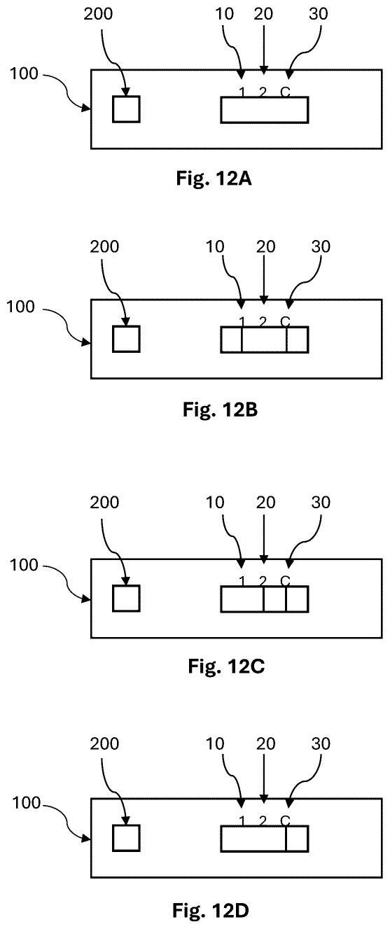

An exemplary lateral flow immunoassay device (100) for L. pneumophila SG1 and SG2-15 is shown in FIGS. 12A-D.

Lateral flow immunoassay device (100) comprises a first test line 10 for L. pneumophila SG1, a second test line 20 for L. pneumophila SG2-15, a control line 30, and a sample pad 200.

The capture antibody for the first test line 10 is a conventional anti-L. pneumophila SG1 antibody (Thermo Fisher Scientific catalog number: MA1-83288).

The capture antibody for the second test line 20 is anti-L. pneumophila SG2-15 antibodies produced as detailed above.

In-use, a patient sample is mixed with running buffer and deposited onto the sample pad 200. Results are then obtained as shown in FIGS. 12B-D, depending on the presence or absence of SG1 and SG2-15 in the patient sample.

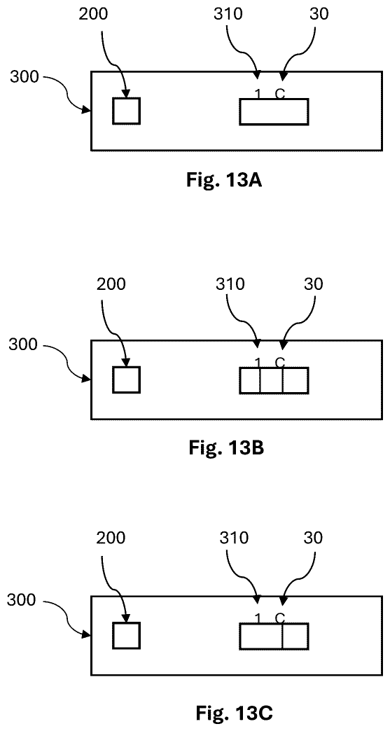

A second lateral flow immunoassay device (300) for L. pneumophila SG1-15 is shown in FIGS. 13A-C.

Lateral flow immunoassay device (300) comprises a first test line 310 for L. pneumophila SG1-15, a control line 30, and a sample pad 200.

The capture antibody for the first test line 310 is a conventional anti-L. pneumophila SG1 antibody (Thermo Fisher Scientific catalog number: MA1-83288) together with anti-L. pneumophila SG2-15 antibodies produced as detailed above.

In-use, a patient sample is mixed with running buffer and deposited onto the sample pad 200. Results are then obtained as shown in FIGS. 12B and 12C, depending on the presence or absence of SG1-15 in the patient sample.

Following from the above description and invention summaries, it should be apparent to those of ordinary skill in the art that, while the methods and apparatuses herein described constitute exemplary embodiments of the present invention, the invention contained herein is not limited to this precise embodiment and that changes may be made to such embodiments without departing from the scope of the invention as defined by the claims. Additionally, it is to be understood that the invention is defined by the claims and it is not intended that any limitations or elements describing the exemplary embodiments set forth herein are to be incorporated into the interpretation of any claim element unless such limitation or element is explicitly stated. Likewise, it is to be understood that it is not necessary to meet any or all of the identified advantages or objects of the invention disclosed herein in order to fall within the scope of any claims, since the invention is defined by the claims and since inherent and/or unforeseen advantages of the present invention may exist even though they may not have been explicitly discussed herein.

### REFERENCE SIGNS

10—first test line
20—second test line
30—control line
100—lateral flow immunoassay device
200—sample pad
300—lateral flow immunoassay device
310—first test line

### Aspects:

Other aspects of the present disclosure include the following:

Aspect 1. An immunogen for the production of anti-Legionella pneumophila serogroups 2-15 antibodies, comprising L. pneumophila serogroups 3, 4, 7, 8, 11, 13, and 15.

Aspect 2. An immunogen according to Aspect 1, wherein the L. pneumophila serogroup 3 is serogroup 3 (Bloomington-2).

Aspect 3. An immunogen according to any preceding Aspect, wherein the L. pneumophila serogroup 4 is serogroup 4 (Los Angeles-1).

Aspect 4. An immunogen according to any preceding Aspect, wherein the L. pneumophila serogroup 7 is serogroup 7 (Chicago-8).

Aspect 5. An immunogen according to any receding Aspect, wherein the L. pneumophila serogroup 8 is serogroup 8 (Concorde-3).

Aspect 6. An immunogen according to any preceding Aspect, wherein the serogroup 15 is serogroup 15 (Lansing-3).

Aspect 7. An immunogen according to any preceding Aspect, wherein it comprises L. pneumophila serogroups 3 (Bloomington-2), 4 (Los Angeles-1), 7 (Chicago-8), 8 (Concorde-3), 11, 13, and 15 (Lansing-3).

Aspect 8. An immunogen according to Aspect 7, wherein it comprises L. pneumophila serogroups 3 (Bloomington-2 NCTC 11232), 4 (Los Angeles-1 NCTC 11233), 7 (Chicago-8 NCTC 11984), 8 (Concorde-3 NCTC 11985), 11 (NCTC 12179), 13 (NCTC 12181), and 15 (Lansing-3 ATCC 35251).

Aspect 9. An immunogen according to any preceding Aspect, wherein the L. pneumophila have been killed.

Aspect 10. An immunogen according to any preceding Aspect, wherein it is for use in the production of anti-Legionella pneumophila serogroups 2-15 antibodies by immunisation.

Aspect 11. A method of producing anti-Legionella pneumophila serogroups 2-15 antibodies comprising collecting antiserum from an animal which has been immunised with an immunogen according to any of Aspects 1-9.

Aspect 12. A method according to Aspect 11, additionally comprising the step of isolating and/or purifying anti-Legionella pneumophila serogroups 2-15 antibodies from the antiserum.

Aspect 13. Anti-Legionella pneumophila serogroups 2-15 antibodies produced according to the method of Aspect 11 or 12 or antigen binding fragments thereof.

Aspect 14. Anti-Legionella pneumophila serogroups 2-15 antibodies or antigen binding fragments thereof according to Aspect 13, in combination with anti-Legionella pneumophila serogroup 1 antibody or antigen binding fragments thereof.

Aspect 15. An immunoassay for detecting the presence of Legionella pneumophila, comprising anti-Legionella pneumophila antibodies or antigen binding fragments thereof according to Aspect 13 or 14.

Aspect 16. An immunoassay according to Aspect 15, wherein it is selected from the group consisting of: lateral flow immunoassay, enzyme-linked immunosorbent assay (ELISA).

Aspect 17. An immunoassay according to Aspect 16, wherein it is a lateral flow immunoassay, and comprises:
a first test line comprising anti-Legionella pneumophila serogroup 1 antibody or an antigen binding fragment thereof;
a second test line comprising anti-Legionella pneumophila serogroups 2-15 antibodies produced according to the method of Aspect 11 or 12 or antigen binding fragments thereof.

Aspect 18. A method of detecting the presence of Legionella pneumophila in a sample, the method comprising the steps of:
(i) contacting the sample with anti-Legionella pneumophila antibodies or antigen binding fragments thereof according to Aspect 13 or 14;
(ii) detecting any antibody-antigen binding reaction; and
(iii) correlating the results of detection step (ii) with the presence of Legionella pneumophila.

1. An immunogen comprising L. pneumophila serogroups, the L. pneumophila serogroups consisting of serogroups 3, 4, 7, 8, 11, 13, and 15.

2. The immunogen of claim 1, wherein the L. pneumophila serogroup 3 is serogroup 3 (Bloomington-2).

3. The immunogen of claim 1, wherein the L. pneumophila serogroup 4 is serogroup 4 (Los Angeles-1).

4. The immunogen of claim 1, wherein the L. pneumophila serogroup 7 is serogroup 7 (Chicago-8).

5. The immunogen of claim 1, wherein the L. pneumophila serogroup 8 is serogroup 8 (Concorde-3).

6. The immunogen of claim 1, wherein the serogroup 15 is serogroup 15 (Lansing-3).

7. The immunogen of claim 1, wherein the L. pneumophila serogroups are serogroups 3 (Bloomington-2), 4 (Los Angeles-1), 7 (Chicago-8), 8 (Concorde-3), 11, 13, and 15 (Lansing-3).

8. The immunogen of claim 1, wherein the L. pneumophila serogroups are serogroups 3 (Bloomington-2 NCTC 11232), 4 (Los Angeles-1 NCTC 11233), 7 (Chicago-8 NCTC 11984), 8 (Concorde-3 NCTC 11985), 11 (NCTC 12179), 13 (NCTC 12181), and 15 (Lansing-3 ATCC 35251).

9. The immunogen of claim 1, wherein the L. pneumophila serogroups are dead.

10. The immunogen of claim 1, for use in the production of antibodies specific against each of Legionella pneumophila serogroups 2-15 by immunization.

11. A method for immunization, the method comprising the step of administering the immunogen of claim 1 to a subject.

12. A method for producing antibodies specific against each of Legionella pneumophila serogroups 2-15, the method comprising collecting antiserum from an animal which has been immunized with the immunogen of claim 1.

13. The method of claim 12, further comprising the step of isolating and/or purifying antibodies specific against each of Legionella pneumophila serogroups 2-15 from the collected antiserum.

14. Antibodies specific against each of Legionella pneumophila serogroups 2-15 or antigen binding fragments thereof, the antibodies produced according to the method of claim 13.

15. Antibodies specific against each of Legionella pneumophila serogroups 2-15 or antigen binding fragments thereof claim 14, in combination with anti-Legionella pneumophila serogroup 1 antibody or an antigen binding fragment thereof.

16. An immunoassay for detecting the presence of Legionella pneumophila, comprising antibodies or antigen binding fragments thereof claim 14.

17. The immunoassay of claim 16, wherein the immunoassay is selected from the group consisting of: a lateral flow immunoassay and/or an enzyme-linked immunosorbent assay (ELISA).

18. An immunoassay according to claim 17, wherein the immunoassay is a lateral flow immunoassay, and comprises:
a first test line comprising antibody specific against Legionella pneumophila serogroup 1 or an antigen binding fragment thereof; and
a second test line comprising antibodies specific against each of Legionella pneumophila serogroups 2-15 or antigen binding fragments thereof, the antibodies produced according to the method of claim 13.

19. A method for detecting the presence of Legionella pneumophila in a sample, the method comprising the steps of:
(i) contacting the sample with antibodies specific against Legionella pneumophila or antigen binding fragments thereof claim 14;
(ii) detecting any antibody-antigen binding reaction; and
(iii) correlating the results of detection step (ii) with the presence of Legionella pneumophila.
